(19) United States
(12) Patent Application Publication
(10) Pub. No.: US 2012/0238606 A1
(43) Pub. Date: Sep. 20, 2012

(54) AZILSARTAN ORGANIC AMINE SALTS, PREPARATION METHOD AND USE THEREOF

(75) Inventors: Aifeng Lv, Jiangsu (CN); Baohai Yang, Jiangsu (CN)

(73) Assignee: JIANGSU HANS PHARMACEUTIAL GROUP CO., LTD., Jiangsu (CN)

(21) Appl. No.: 13/512,652

(22) PCT Filed: Nov. 29, 2010
(86) PCT No.: PCT/CN10/79222

§ 371 Date: May 31, 2012

(30) Foreign Application Priority Data
Nov. 30, 2009 (CN) 200910246554.9

(51) Int. Cl. C07D 413/10; A61P 9/12; A61K 31/4245

(52) U.S. Cl. 514/364; 548/132

(57) ABSTRACT

Azilsartan amine salts, their preparation and method of use are disclosed. Specifically, azilsartan amine salts represented by formula (I), their preparation, pharmaceutical compositions containing a therapeutically effective amount of the compounds and their use for treating hypertension are disclosed.

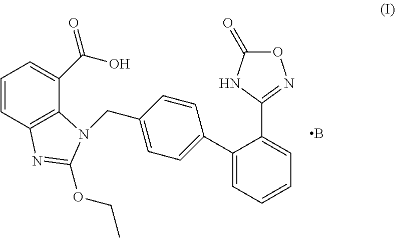

### FIELD OF THE INVENTION

The present invention relates to azilsartan amine salts, preparation method thereof, and pharmaceutical compositions containing a therapeutically effective amount of the compounds and their use as antihypertensive medicaments.

### BACKGROUND OF THE INVENTION

Azilsartan is a selective antagonist of the hypertension II1 receptor (AT1). It reduces blood pressure by selectively blocking the binding of hypertension II to the receptor of vascular smooth muscle AT1, which stops a vein from shrinking induced by hypertension II.

There is carboxyl group in the molecular structure of azilsartan, which leads to bad absorption of azilsartan in vivo. Azilsartan, therefore, is difficult to deliver in pharmaceutical dosage forms. In order to improve its bioavailability, azilsartan is converted to an ester by chemical modification, but its bioavailability is still not satisfactory, and its molecular structure becomes complex by such modification, which increases difficulty in syntheses.

### DETAILED DESCRIPTION OF THE INVENTION

The present invention provides salts formed from azilsartan and amines that have better pharmacokinetic character, higher bioavailability. The salts are also more suitable for preparation processes.

The present invention provides for azilsartan amine salts represented by formula (I):

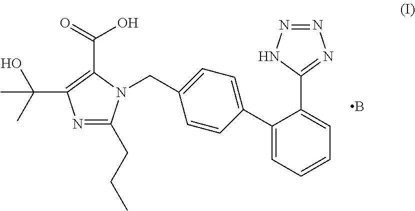

wherein, B is an amine selected from methylamine, dimethylamine, trimethylamine, ethylamine, diethylamine, triethylamine, ethanolamine, piperazidine, dibenzylethylenediamine, meglumine, tromethamine, tetramethyl quaternary ammonium, tetraethyl quaternary ammonium or choline; preferably ethanolamine

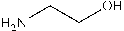

or choline

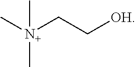

The present invention also provides a process for preparing the compounds described above, which comprises adding an acid form of azilsartan and amine B separately into organic solvents, such as alcohol, to obtain corresponding salts at room temperature or under heating, wherein the solvents are selected from the group consisting of methanol, ethanol, propanol or isopropanol.

The present invention also provides a pharmaceutical composition for use in the treatment of hypertension, comprising a therapeutically effective amount of azilsartan amine salts as an active ingredient and pharmaceutically acceptable carriers.

Furthermore, the invention also provides application of azilsartan amine salts and their pharmaceutical compositions in the preparation of an antihypertensive medicament.

In the preparation process of pharmaceutical compositions, it is important to provide a drug into an appropriate dosage form which is not only commercially viable, but also pharmaceutically appropriate.

In another aspect, it is important to provide a reliable, reproducible dosage form resulting in a fairly constant drug plasma concentration curve after administrating to a subject.

Other important factors to be considered include chemical durability, solid-state stability, and storage life of the active ingredient. The drugs and the compositions containing them can be preferably stored relatively for a long time with no obvious change in physical and chemical properties of the active components such as chemical composition, density, hygroscopicity and solubility.

The purity of the drug can also be important.

If a drug can be obtained in a stable form such as a stable crystal form, then the drug can usually offer the following advantages: convenient handling, easy preparation, appropriate drug dosage forms and reliable solubility.

An effective amount of the active ingredient in a pharmaceutical dosage unit as described above will be nontoxic, preferably selected from the range 0.001-100 mg/kg of total weight, more preferably 0.001-50 mg/kg. When treating a subject with an azilsartan amine salt, the selected dose is administrated preferably orally or parenterally. Preferred parenteral forms include topical, rectal, transdermal administration forms, injection and continuous infusion. Oral dosage units for human administration preferably contain from 0.05 to 3500 mg of active ingredient, most preferably from 0.5 to 1000 mg of active ingredient. Oral administration, which uses a lower dosage, is more preferred. Parenteral administration, at high dosages, however, also can be used when safe and convenient for the patient. The above dosages relate to the preferred amount of the active ingredient as the free acid.

It will be understood by one skilled in the art that the optimal quantity and period of dosages of the active ingredient for an patient will depend on the nature and extent of the condition of the particular patient to be treated, the form, route and site of administration, and such optimums can be determined by conventional techniques. It will also be appreciated by one skilled in the art that the optimal course of treatment, i.e., the number of doses of the active ingredient given per day for a defined number of days, can be ascertained by those skilled in the art using conventional testing methods.

The compounds of the present invention can be administrated orally or parenterally, wherein the compounds can be prepared into tablets, pills, powder and granules used in different routes of administration. In above solid dosage forms, the active components mixed with at least one kind of inert diluent. According to conventional operation, oral dosage forms also include other substance such as lubricants, glidants and antioxidants besides inert diluent. If made into capsules, tablets and pills, dosage forms contain buffering agents. Tablets and pills can be made into sustained-release dosage forms, too.

Although non-aqueous emulsions can be used, parenteral dosage forms containing a sterile aqueous solution can also be used. These dosage forms may also contain adjuvants, for example antiseptics, wetting agents, penetrating agents, buffering agents, emulsifying agents and dispersants. The sterilizing process can performed using a bacteria retaining filter, sterilizing agents added to the compositions, irradiation or heating.

Compared with Azilsartan and esters of Azilsartan, the salts of the present invention mainly have the following advantages:

(1) The salts of the present invention are easily dissolved in conventional solvents such as water, methanol, 0.1% hydrochloric acid and adapted to be prepared into conventional dosage forms.

(2) The salts of the present invention have improved stability.

(3) The salts of the present invention have better bioavailability.

(4) The preparation process of the salts of the present invention has the advantages of high yield, high purity, quick, convenience and low cost. In some embodiments, the ethanolamine and choline salts can be more advantageous in preparation process routes.

### PREFERRED EMBODIMENTS

### EXAMPLE 1

### Preparation of Azilsartan ethanolamine

Azilsartan (acid form) (1.37 g) was added into methanol (30 ml), then to the mixture ethanolamine (0.183 g) was added at room temperature and the resulting mixture was heated to reflux. The insoluble substance was filtered out, and the filtrate was concentrated under reduced pressure to remove solvent. Acetone (20 ml) was added into the residue, then the mixture was stirred for 2 hours, filtered, dried to obtain white solid 1.45 g Azilsartan ethanolamine.

1H NMR (DMSO-d6+D₂O) data: δ: 1.35 (t, 3H, CH₃), 2.83 (t, 2H, CH₂), 3.55 (t, 2H, CH₂), 4.52 (q, 2H, CH₂), 5.65 (s, 2H, CH₂), 7.03˜7.51 (m, 11H).

### EXAMPLE 2

### Preparation of Azilsartan choline

Azilsartan (1.37 g) and 46% aqueous solution of choline (0.79 g) were added into ethanol (20 ml) and the mixture was heated to reflux for 2 hours, and then stirred for one day at room temperature. The mixture was dried under reduced pressure to remove the solvents. Ethyl acetate (20 ml) was added, then the mixture was stirred for 2 hours, and filterated to obtain white solid azilsartan choline.

1H NMR (DMSO-d6+D2O) data: δ: 1.35 (t, 3H, CH₃), 3.83 (t, 2H, CH₂), 3.55 (t, 2H, CH₂), 3.06 (s, 9H), 4.52 (q, 2H, CH₂), 5.65 (s, 2H, CH₂), 7.05˜7.50 (m, 11H).

### EXPERIMENTAL EXAMPLE 1

The effect of compounds of the present invention on blood pressure in angiotensin II-induced hypertensive rats was investigated.

Male Sprague-Dawley rats (9-11 weeks old, CLEA Japan, Inc.) were anesthetized by pentobarbital solution (50 mg/kg, ip.) and prepared for surgery. The surgery procedure was as follows: made an incision through the skin on the abdomen and abdominal wall, separated the aorta and vein from the vena cava, occluded blood flow to allow introduction of a polyethylene (PE) tube filled with normal saline containing heparin (200 U/mL) into the vessel, passed the PE tube through the subcutaneous slits, and fixed the PE tube at the back of the neck. After recovery period, the rats were induced hypertension by intravenous administration of 100 ng/kg angiotensin II (AII). The PE tube was combined with a pressure transducer coupled to a blood pressure monitor amplifier (2238, NEC San-ei Instruments). The animals were enrolled based on average systolic blood pressure over 24 hours. All the animals with average systolic blood pressure less than 140 mmHg were excluded from this study, and the others were divided into 2 groups. The groups were single oral dosed equimolar amount of test articles. The rats were injected intravenously AII again after 24 hours, and their blood pressure were measured by the monitor. The rat blood pressure inhibition ratios after dosing were calculated. The test articles were formulated in 0.5% methyl cellulose, and the dosing volume for all animals was 2 mL/kg. The results were expressed as Mean±S.E. (Table 1).

TABLE 1

| | | Dose | | |
|---|---|---|---|---|
| Test article | n | (mg/kg) | Route | Inhibition ratios (%) |
| Product of example 1 | 4 | 0.11 | ig | 57.4 ± 6.7 |
| Product of example 2 | 6 | 0.123 | ig | 54.3 ± 5.1 |

Results of the experiment: The blood pressure significantly decreased following the treatments of present invention compounds, and the inhibition could persist for an extended period of time.

### EXPERIMENTAL EXAMPLE 2

The effect of compounds of the present invention on blood pressure in angiotensin II-induced hypertensive dogs was investigated.

In this study, male Beagle dogs (weighting 12.0-14.7 kg, KITAYAMA LABES, CO., LTD.) were employed. The dogs were anesthetized by pentobarbital solution (50 mg/kg, ip) and prepared for surgery. The surgery procedure was as follows: made an endotracheal intubation for controlling the breathing, shaved the regions of femoral and back of the neck, fixed the dogs at the dorsal position, used isodine solution (MEIJI SEIKA KAISHA, LTD.) to sterilize the skins, made an incision through the skin on the right femoral region, separated the arteria femoralis, occluded blood flow to allow introduction of a mirror catheter (5 F, MILLER INDUSTRIES) and polyurethane tube into the aorta and vein, respectively, passed the tubes through the subcutaneous slits and fixed the tubes at the back, closed the wall and sutured the skin, and injected intramuscularly the penicillin G potassium (MEIJI SEIKA KAISHA, LTD., 40,000 units) for sterilization. For post-operative care, animals were administered 40,000 units penicillin G potassium intramuscularly once daily for 3 days. There was a recovery period before study ongoing.

The animals were singly housed in a small metabolic cage and fasted during the experiment. The mirror catheter was linked to a transducer unit (MILLER INDUSTRIES). Through a DC amplifier (N4777, NEC San-ei Instruments) and a blood pressure monitor amplifier (N4441, NEC San-ei Instruments), the blood pressure was recorded by a recorder (RECTI-HORIZ 8 K, NEC San-Ei Instruments). In order to establish a hypertension model, the dogs were injected intravenously 100 ng/kg AII for 3 or 4 times before dosing test articles. The test articles were an equal molar dose suspended in 0.5% methyl cellulose, and the dosing volume for all animals was 2 mL/kg. After administration, the blood pressure was measured by the monitor. The dog blood pressure inhibition ratios after dosing were calculated, and the results were expressed as mean±S.E. (Table 2).

TABLE 2

| | | Dose | | Inhibition ratios (%) | |
|---|---|---|---|---|---|
| Test article | n | (mg/kg) | Route | 10 hr post dosing | 24 hr post dosing |
| Product of | 6 | 1.10 | ig | 82.7 ± 7.3 | 61.6 ± 4.8 |
| example 1 | | | | | |
| Product of | 5 | 1.23 | ig | 79.3 ± 8.6 | 59.5 ± 6.3 |
| example 2 | | | | | |

Results of the experiment: The blood pressure significantly decreased following the compounds of the present invention treatments. Also, the inhibitory effect sustained for an extended period of time.

1. Azilsartan amine salts represented by formula (I): 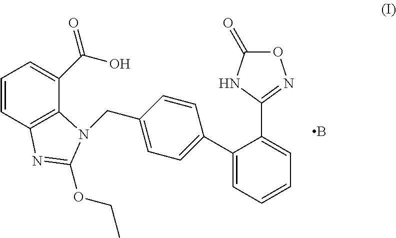
wherein, B is an amine.

2. The azilsartan amine salts of claim 1, wherein B is selected from the group consisting of: methylamine, dimethylamine, trimethylamine, ethylamine, diethylamine, triethylamine, ethanolamine, piperazidine, dibenzylethylenediamine, meglumine, tromethamine, tetramethyl quaternary ammonium, tetraethyl quaternary ammonium and choline.

3. The azilsartan amine salts of claim 1, wherein B is ethanolamine represented by formula (II)

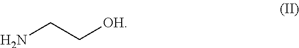

4. The azilsartan amine salts of claim 1, wherein B is choline represented by formula (III)

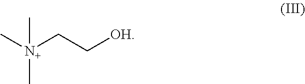

5. A process for preparing the azilsartan amine salts of any one of claims 1 to 4, which comprises adding azilsartan and amine B separately into one or more alcohol solvents to obtain corresponding salts at room temperature or under heating.

6. The process of claim 5, wherein the alcohol solvents are selected from the group consisting of: methanol, ethanol, propanol and isopropanol.

7. A pharmaceutical composition, comprising a therapeutically effective amount of the azilsartan amine salts of any one of claims 1 to 4 as an active ingredient and a pharmaceutically acceptable carrier.

8. A method of treating hypertension, comprising administering an azilsartan amine salts of any one of claims 1 to 4 to a patient.

9. A method of treating hypertension, comprising administering a pharmaceutical composition of claim 7 to a patient.
